(19) United States
(12) Patent Application Publication
(10) Pub. No.: US 2025/0109102 A1
(43) Pub. Date: Apr. 3, 2025

(54) METHOD FOR PREPARING (2,2,2-TRIFLUOROETHYL)SULFANYLANILINE DERIVATIVES

(71) Applicant: BAYER AKTIENGESELLSCHAFT, Leverkusen (DE)

(72) Inventors: ALEXANDER ARLT, Köln (DE); DIRK BROHM, Mettmann (DE); THOMAS HIMMLER, Odenthal (DE)

(21) Appl. No.: 18/291,287

(22) PCT Filed: Jul. 22, 2022
(86) PCT No.: PCT/EP2022/070624

§ 371 Date: Jan. 23, 2024

(30) Foreign Application Priority Data
Jul. 27, 2021 (EP) 21187944.0

(51) Int. Cl. C07C 323/36

(52) U.S. Cl. CPC C07C 323/36

(57) ABSTRACT

The present invention relates to a method for preparing (2,2,2-trifluoroethyl)sulfanylaniline derivatives of the formula (I),

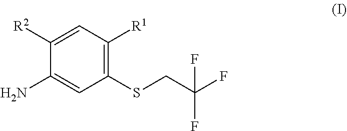

in which R¹ and R² have the definitions specified in the description.

The present invention relates to a method for preparing (2,2,2-trifluoroethyl)sulfanylaniline derivatives.

(2,2,2-Trifluoroethyl)sulfanylaniline derivatives are of great importance in the agrochemical industry as intermediates for the synthesis of active ingredients. There is therefore an ongoing need for simplified, technically and economically feasible methods for their synthesis.

It is known that (2,2,2-trifluoroethyl)sulfanylaniline derivatives can be obtained by alkylation of a thiophenol with 1,1,1-trifluoro-2-iodoethane (e.g. WO2014202505) or with bis(2,2,2-trifluoroethyl) sulfate (Chem. Sci., 2019, 10, 10331-10335). It is also possible to replace 1,1,1-trifluoro-2-iodoethane by 2,2,2-trifluoroethyl methanesulfonate.

In addition, the alkylation of thiophenols with 1,1,1-trifluoro-2-chloroethane has been described in patent application EP 0645355. 1,1,1-Trifluoro-2-chloroethane, known to be very slow-reacting, was reacted with aliphatic, aromatic and heterocyclic thiolates. The strong bases sodium hydride or an aqueous sodium hydroxide solution were used as base to prepare the thiolates from the thiols. The polar aprotic solvent dimethylformamide was used exclusively as solvent.

Dimethylformamide is a very polar aprotic solvent. It is therefore particularly used as solvent for nucleophilic substitution reactions. However, due to its toxicological properties, it is classified as teratogenic and its use should be reduced to the level absolutely necessary.

The choice of solvent used in a production process, besides these toxicological aspects, depends on many other factors, such as the solubility of reactants and products, the influence on the activity of the reactants, the stability of the solvent under the reaction conditions, the influence on the undesired secondary components formed and the costs as well as the availability of an appropriate production site. Although, in principle, there may be various suitable possibilities, the choice of a suitable solvent or a suitable solvent mixture is not a trivial task for the reasons cited above.

For chemical and/or economical reasons, it may also make sense not to change the solvent for the individual chemical steps in a multi-stage synthetic sequence. In this case, a solvent or a solvent mixture has to be identified which meets the aforementioned requirements in two different reactions.

Thiophenols are well known to be sensitive to oxidation. Under the influence of atmospheric oxygen, disulfides form by an oxidative dimerization reaction. These disulfides are no longer available for alkylation by electrophiles and therefore reduce the yield considerably. In addition, these disulfides are an impurity which subsequently may have to be laboriously removed. The more electron-rich the thiophenol, also the more sensitive it is to oxidation.

The substituted 3-aminobenzenethiols required for the preparation of (2,2,2-trifluoroethyl)sulfanylaniline derivatives (I) are very sensitive to oxidation owing to the electron-rich 3-amino function and must therefore be handled under an inert gas atmosphere. It would therefore be highly advantageous if these substituted 3-aminobenzenethiols did not have to be isolated after their preparation. This would significantly reduce the risk of oxidation of these intermediates. The failure rate of the production process would therefore be reduced.

The 3-aminobenzenethiols required for the preparation of (2,2,2-trifluoroethyl)sulfanylaniline derivatives (I) can be obtained from 1,1′-disulfandiylbis (3-nitrobenzene) derivatives by a transition metal-catalysed reduction with hydrogen. It has been found that this reduction takes place advantageously in the solvents THF or ethyl acetate (WO2014/090913). However, alkylation with 1,1,1-trifluoro-2-chloroethane to give (2,2,2-trifluoroethyl)sulfanylaniline is only described with dimethylformamide as solvent.

Consequently, taking into account the prior art outlined, there existed an ongoing need for a simplified, technically and economically feasible method for alkylation of substituted 3-aminobenzenethiols, especially 5-amino-4-fluoro-2-methylbenzenethiol. The method envisaged should make it possible to obtain the desired target compounds starting from solutions of substituted 3-aminobenzenethiols in non-polar solvents, particularly in THE or ethyl acetate, and avoid or at least significantly reduce the use of polar solvents such as dimethylformamide. The (2,2,2-trifluoroethyl)sulfanylaniline derivatives obtainable by this desired method should preferably in this case be obtained in high yield and at high chemical purity.

Surprisingly, it has now been found that the alkylation of 3-aminobenzenethiols with 1,1,1-trifluoro-2-chloroethane to give (2,2,2-trifluoroethyl)sulfanylaniline derivatives in solvent mixtures of a first, polar solvent, such as dimethylformamide or dimethylacetamide, with a second, significantly less polar solvent, such as ethyl acetate or tetrahydrofuran, proceeds in good yields. This is all the more surprising given that, due to the low reactivity of 1,1,1-trifluoro-2-chloroethane, those skilled in the art would have expected the reaction to cease when lowering the polarity of the solvent or solvent mixture. It is known that the polarity of the solvent has a major influence on nucleophilic substitution reactions such as the alkylation reaction reported here. In addition, it was not foreseeable that it would even be possible to use a mixture in which the polar solvent is present in deficiency.

The present invention accordingly relates to a method for preparing (2,2,2-trifluoroethyl)sulfanylaniline derivatives of the formula (I):

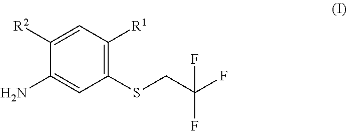

in which R¹ and R² are each independently (C₁-C₃)-alkyl or halogen,
which is characterized in that a 3-aminobenzenethiol of the formula (II):

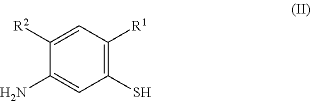

in which R¹ and R² have the definitions given above,
is reacted with 1,1,1-trifluoro-2-chloroethane in the presence of a base in a solvent mixture, wherein the solvent mixture comprises
   (i) a first (polar aprotic) solvent selected from N-methylpyrrolidone, N-ethylpyrrolidone, N-methylformamide, dimethylformamide, N,N-dimethylacetamide (DMAc), 1,3-dimethyl-2-imidazolidinone, tetramethylurea, sulfolane, dimethyl sulfoxide, acetonitrile, propionitrile, butyronitrile, polyethylene glycols, ethylene carbonate and propylene carbonate, and
   (ii) a second (less polar aprotic) solvent selected from tetrahydrofuran (THF), 1,2-dimethoxyethane (DME), 1,4-dioxane, diethyl ether, methyl tertbutyl ether (MTBE), tert-amyl methyl ether (TAME), 2-methyl-THF, cyclopentyl methyl ether, bis(2-methoxyethyl) ether, anisole, ethyl acetate, isopropyl acetate, butyl acetate, pentyl acetate, 3,3-dimethylbutanone, diethyl carbonate, dimethyl carbonate, toluene, xylenes and ethylbenzene.

In a further embodiment of the invention, the solvent mixture comprises
(i) a first (polar aprotic) solvent selected from N-methylpyrrolidone, N-ethylpyrrolidone, N-methylformamide, dimethylformamide, N,N-dimethylacetamide (DMAc), 1,3-dimethyl-2-imidazolidinone, tetramethylurea, sulfolane, dimethyl sulfoxide, acetonitrile, propionitrile, butyronitrile, ethylene carbonate and propylene carbonate, and
(ii) a second (less polar aprotic) solvent selected from tetrahydrofuran (THF), 1,2-dimethoxyethane (DME), 1,4-dioxane, diethyl ether, methyl tertbutyl ether (MTBE), tert-amyl methyl ether (TAME), 2-methyl-THF, cyclopentyl methyl ether, bis(2-methoxyethyl) ether, anisole, ethyl acetate, isopropyl acetate, butyl acetate, pentyl acetate, 3,3-dimethylbutanone, toluene, xylenes and ethylbenzene.

Preferred, particularly preferred and especially preferred definitions of the radicals R¹ and R² specified in the aforementioned formulae (I) and (II) are elucidated below.

R¹ and R² are preferably each independently fluorine, chlorine or methyl.

R¹ and R² are particularly preferably each independently fluorine or methyl.

With emphasis, R¹ is methyl and R² is fluorine.

Surprisingly, the (2,2,2-trifluoroethyl)sulfanylaniline derivatives of the formula (I) can be prepared by the method of the invention in good yields. Furthermore, the method according to the invention allows the use of solvent mixtures of a polar solvent with a significantly less polar solvent suitable for an industrial scale.

The method according to the invention can be illustrated by the following scheme (1):

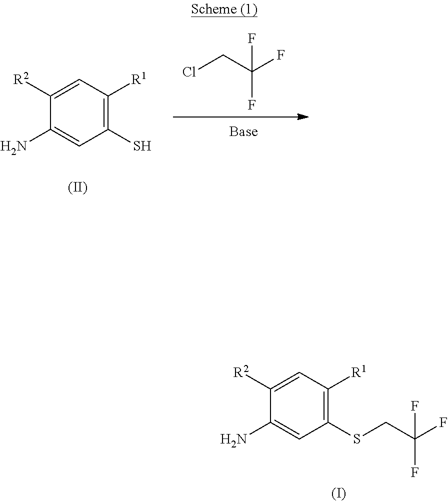

In scheme (1), R¹ and R² have the definitions specified above.

The required substituted 3-aminobenzenethiols of the formula (II) may be obtained by analogy to the method described, for example, in WO2014/090913.

The method may also be carried out with derivatives of 3-aminobenzenethiols in which one or both protons of the amino group has/have been substituted by-CO (C₁-C₆) alkyl (alkanoyl) or —SO₂(C₁-C₆) alkyl (alkylsulfonyl).

### General Definitions

In the context of the present invention, the term “halogens” (Hal) encompasses, unless otherwise defined, elements selected from the group consisting of fluorine, chlorine, bromine and iodine, preference being given to using fluorine, chlorine and bromine, and particular preference to using fluorine and chlorine.

Optionally substituted groups may be singly or multiply substituted; if multiply substituted, the substituents may be identical or different. Unless otherwise stated at the relevant position, the substituents are selected from halogen, (C₁-C₆) alkyl, (C₃-C₁₀) cycloalkyl, cyano, nitro, hydroxy, (C₁-C₆) alkoxy, (C₁-C₆) haloalkyl and (C₁-C₆) haloalkoxy, in particular from fluorine, chlorine, (C₁-C₃) alkyl, (C₃-C₆) cycloalkyl, cyclopropyl, cyano, (C₁-C₃) alkoxy, (C₁-C₃) haloalkyl and (C₁-C₃) haloalkoxy.

Alkyl groups substituted by one or more halogen atoms (Hal) are, for example, selected from trifluoromethyl (CF₃), difluoromethyl (CHF₂), CF₃CH₂, ClCH₂ or CF₃CCl₂.

Alkyl groups in the context of the present invention are, unless otherwise defined, linear, branched or cyclic saturated hydrocarbon groups.

The definition C₁-C₃-alkyl encompasses the widest range defined herein for an alkyl group. In detail, this definition encompasses, for example, methyl, ethyl, n-propyl, isopropyl.

The substituted 3-aminobenzenethiols of the formula (II) are reacted to give compounds of the formula (I) in the presence of a solvent mixture. This solvent mixture comprises a first and a second solvent. In a further configuration, this solvent mixture consists of the first and the second solvent.

The first solvent is a polar aprotic solvent and the second solvent is a less polar aprotic solvent. Such solvents are referred to below.

In the context of the present application, the first and thus polar aprotic solvents are: N-methylpyrrolidone, N-ethylpyrrolidone, N-methylformamide, dimethylformamide, N,N-dimethylacetamide (DMAc), 1,3-dimethyl-2-imidazolidinone, tetramethylurea, sulfolane, dimethyl sulfoxide, acetonitrile, propionitrile, butyronitrile, polyethylene glycols, ethylene carbonate and propylene carbonate.

In the context of the application, the second and thus less polar aprotic solvents are: tetrahydrofuran (THF), 1,2-dimethoxyethane (DME), 1,4-dioxane, diethyl ether, methyl tertbutyl ether (MTBE), tert-amyl methyl ether (TAME), 2-methyl-THF, cyclopentyl methyl ether, bis(2-methoxyethyl) ether, anisole, ethyl acetate, isopropyl acetate, butyl acetate, pentyl acetate, 3,3-dimethylbutanone, diethyl carbonate, dimethyl carbonate, toluene, xylenes and ethylbenzene.

Preferred first and thus polar aprotic solvents are: N-methylpyrrolidone, N-methylformamide, dimethylformamide, N,N-dimethylacetamide (DMAc), sulfolane, dimethyl sulfoxide and polyethylene glycols of molar masses of 200-800 g/mol (polyethylene glycol 200-800).

Likewise preferred first and thus polar aprotic solvents are: N-methylpyrrolidone, N-methylformamide, dimethylformamide, N,N-dimethylacetamide (DMAc), sulfolane and dimethyl sulfoxide.

Preferred second and thus less polar aprotic solvents are: tetrahydrofuran (THF), dimethyl ether (DME), 1,4-dioxane, 2-methyl-THF, ethyl acetate, isopropyl acetate, butyl acetate and pentyl acetate.

Particularly preferred first and thus polar aprotic solvents are: N-methylpyrrolidone, dimethylformamide, N,N-dimethylacetamide (DMAc), dimethyl sulfoxide and polyethylene glycol 400.

Likewise particularly preferred first and thus polar aprotic solvents are: N-methylpyrrolidone, dimethylformamide and N,N-dimethylacetamide (DMAc).

Particularly preferred second and thus less polar aprotic solvents are: tetrahydrofuran (THF), ethyl acetate and isopropyl acetate.

Especially preferred first and thus polar solvents are: dimethylformamide, N,N-dimethylacetamide (DMAc), dimethyl sulfoxide and polyethylene glycol 400.

Likewise especially preferred first and thus polar aprotic solvents are: dimethylformamide and N,N-dimethylacetamide (DMAc).

Especially preferred second and thus less polar aprotic solvents are: tetrahydrofuran (THF) and ethyl acetate.

The ratio of first (polar aprotic) solvent to second (less polar aprotic) solvent is in the range from 20:1 to 1:20, preferably in the range from 2:1 to 1:10, particularly preferably in the range from 1:2 to 1:5 and especially preferably in the range from 1:2 to 1:4, ideally from 1:2 to 1:3.

In alternative configurations, the ratio of first (polar aprotic) solvent to second (less polar aprotic) solvent is in the range from 1:1 to 1:10 or in the range from 1:1 to 1:5 or in the range from 1:1 to 1:3 or in the range from 1:1 to 1:2, or in the range from 2:1 to 1:5 or in the range from 2:1 to 1:3 or in the range from 2:1 to 1:2 or in the range from 1:2 to 1:10 or in the range from 1:2 to 1:20.

The bases that may be used for this reaction are not subject to any particular limitations. Suitable as bases for preparing the thiolates are monobasic or dibasic organic or inorganic bases, preferably in equimolar amounts, such as alkali metal hydroxide, alkaline earth metal hydroxide, ammonium or alkylammonium hydroxide, sodium hydride, calcium hydride, alkali metal or alkaline earth metal alkoxide, alkali metal or alkaline earth metal carbonates, ammonia, primary, secondary or tertiary alkyl, aryl or aralkylamines, amidines or pyridine. Preferred bases are sodium and potassium hydroxide and sodium and potassium carbonate.

Particular preference is given to sodium carbonate and potassium carbonate.

Emphatic preference is given to potassium carbonate.

The bases in this case may be used anhydrous and also as aqueous solutions.

The molar ratio of base to thiol of the formula (II) is in the range from 0.9:1 to 5:1, preferably between 1.1:1 and 2:1.

The reaction is generally carried out at a temperature between 0° C. and 100° C., preferably between 20° C. and 100° C., very particularly preferably between 40° C. and 80° C.

The reaction is typically carried out at standard pressure up to moderate positive pressure, but may also be carried out at a higher positive pressure. Preferred pressure ranges are between 0 bar and 20 bar above atmospheric pressure, in particular between 0 bar and 18 bar above atmospheric pressure, preferably between 0 bar and 15 bar above atmospheric pressure, especially preferably between 0 bar and 10 bar above atmospheric pressure. The positive pressure may be caused by the autogenous pressure of the 1,1,1-trifluoro-2-chloroethane used or by pressure of an additional inert gas such as argon or nitrogen. The reaction may be carried out, for example, in a pressurized autoclave, but does not necessarily have to be carried out in a pressurized autoclave. Various alternatives are known to those skilled in the art in this respect.

The reaction may be carried out in the presence of a phase transfer catalyst, such as tetra-n-butylammonium bromide.

The desired compounds of the formula (I) may be isolated for example by subsequent extraction and distillation.

The present invention is elucidated in detail by the examples that follow, although the examples should not be interpreted in such a manner that they restrict the invention.

### PREPARATION EXAMPLES

The reported yields were calculated by weighing the amount of product obtained and correcting this weight by the purity determined by HPLC in area percent. The proportion of desired product in HPLC area percent was assessed at a wavelength of 210 nm.

In some examples, the amount of product was determined by weighing a solution of the product and correcting this weight by the purity determined by HPLC in percent by weight. The proportion of the target product in the product solution in this case was determined against an external standard. A sample of 2-fluoro-4-methyl-5-[(2,2,2-trifluoroethyl)sulfanyl]aniline of known purity served as external standard.

### Example 1: Synthesis of 2-fluoro-4-methyl-5-[(2,2,2-trifluoroethyl)sulfanyl]aniline in a solvent mixture of dimethylformamide and ethyl acetate (ratio 1:2)

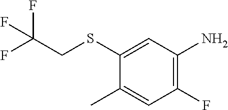

In a 300 mL autoclave composed of Hastelloy alloy were initially charged 8.50 g (92.5% purity, 50.0 mmol) of 5-amino-4-fluoro-2-methylbenzenethiol as a solution in a mixture of 50 ml of ethyl acetate and 25 mL of dimethylformamide. 806 mg (2.50 mmol) of tetra-n-butylammonium bromide and 9.67 g (70.0 mmol) of potassium carbonate were added. The autoclave was then cooled with dry ice and 7.7 g (65 mmol) of 1,1,1-trifluoro-2-chloroethane were introduced. The autoclave was sealed and the internal pressure was increased to 10 bar by introducing argon. The mixture was heated to 60° C. and stirred at this temperature for 16 h (stirring speed: 600 rpm). The autoclave was depressurized and the contents poured into 200 mL of ice water with stirring. The mixture was stirred for 30 min and then the phases were separated. The aqueous phase was extracted three times in total with in each case 150 mL of methyl tert-butyl ether. The combined organic phases were washed twice with 30 mL of water each time and then with 30 ml of saturated sodium chloride solution. The organic phase was dried with a drying agent (sodium sulfate or magnesium sulfate). The drying agent was removed by filtration and the solvent was removed under reduced pressure. 12.3 g of 2-fluoro-4-methyl-5-[(2,2,2-trifluoroethyl)sulfanyl]aniline were obtained as a brown oil at a purity by HPLC of 80.4 area percent and in a yield of 83%.

¹H-NMR (400 MHZ, DMSO-D6): δ=6.97 (d, J=8.8 Hz, 1H), 6.93 (d, J=12.0 Hz, 1H), 5.06 (s, 2H), 3.70 (q, J=10.4 Hz, 2H), 2.24 (s, 3H) ppm.

Under otherwise identical reaction conditions, when replacing dimethylformamide with dimethylacetamide, a yield of 12.60 g of 2-fluoro-4-methyl-5-[(2,2,2-trifluoroethyl)sulfanyl]aniline was obtained at a purity by HPLC of 84.0 area percent and in a yield of 89%.

### Example 2: Synthesis of 2-fluoro-4-methyl-5-[(2,2,2-trifluoroethyl)sulfanyl]aniline in a solvent mixture of dimethylformamide and tetrahydrofuran (ratio 1:2)

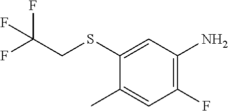

In a 300 mL autoclave composed of Hastelloy alloy were initially charged 8.70 g (90.4% purity, 50.0 mmol) of 5-amino-4-fluoro-2-methylbenzenethiol as a solution in a mixture of 50 ml of tetrahydrofuran and 25 mL of dimethylformamide. 9.67 g (70.0 mmol) of potassium carbonate were added. The autoclave was then cooled with dry ice and 7.7 g (65 mmol) of 1,1,1-trifluoro-2-chloroethane were introduced. The autoclave was sealed and the internal pressure was increased to 10 bar by introducing nitrogen. The mixture was heated to 40° C. and stirred at this temperature for 16 h (stirring speed: 600 rpm). The autoclave was depressurized and the reaction mixture was concentrated under reduced pressure. To the residue was added a mixture of water and methyl tert-butyl ether. The phases were separated and the aqueous phase was extracted with methyl tert-butyl ether. The combined organic phases were washed with water and dried with a drying agent (sodium sulfate or magnesium sulfate). The drying agent was removed by filtration and the solvent was removed under reduced pressure. 12.5 g of 2-fluoro-4-methyl-5-[(2,2,2-trifluoroethyl)sulfanyl]aniline were obtained as an orange oil at a purity by HPLC of 89.8 area percent and in a yield of 94%.

### Example 3: Synthesis of 2-fluoro-4-methyl-5-[(2,2,2-trifluoroethyl)sulfanyl]aniline in a solvent mixture of dimethylformamide and ethyl acetate (ratio 1:2) under autogenous pressure

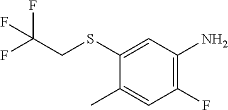

In a 500 mL autoclave composed of Hastelloy alloy were initially charged 23.58 g (150.0 mmol) of 5-amino-4-fluoro-2-methylbenzenethiol, 29.0 g (210 mmol) of potassium carbonate, 2.42 g (7.51 mmol) of tetra-n-butylammonium bromide, 150 ml of ethyl acetate and 75 mL of dimethylformamide. The autoclave was sealed, flushed several times with nitrogen, cooled to −10° C. and at this temperature 23.30 g (196.6 mmol) of 1,1,1-trifluoro-2-chloroethane were introduced. The autoclave was then heated to 60° C. and stirred at this temperature for 15 h (stirring speed: 300 rpm). In this case, the internal pressure increased to 0.7 bar. The mixture was then cooled to 20° C. and the autoclave depressurized. 200 ml of water were added to the reaction mixture, which was stirred for a further 50 min, transferred to a separating funnel and the phases were separated. 174.8 g of an upper phase and 305.6 g of a lower phase were obtained. The proportion of 2-fluoro-4-methyl-5-[(2,2,2-trifluoroethyl)sulfanyl]aniline in the upper phase was determined by quantitative HPLC (against external standard) as 19.3%. This corresponds to a yield of 94% starting from 5-amino-4-fluoro-2-methylbenzenethiol.

### Example 4: Synthesis of 2-fluoro-4-methyl-5-[(2,2,2-trifluoroethyl)sulfanyl]aniline in a solvent mixture of polyethylene glycol 400 and ethyl acetate (ratio 1:2)

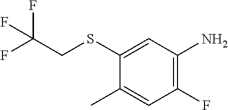

In a 500 mL autoclave composed of Hastelloy alloy were initially charged 23.58 g (150.0 mmol) of 5-amino-4-fluoro-2-methylbenzenethiol, 29.0 g (210 mmol) of potassium carbonate, 2.42 g (7.51 mmol) of tetra-n-butylammonium bromide, 150 ml of ethyl acetate and 75 mL of polyethylene glycol 400. The autoclave was sealed, flushed several times with nitrogen, cooled to −10° C. and at this temperature 24.70 g (208.5 mmol) of 1,1,1-trifluoro-2-chloroethane were introduced. Then, the internal pressure was increased to 4 bar by introducing nitrogen, the autoclave heated to 60° C. and the mixture stirred at this temperature for 63 h (stirring speed: 300 rpm). In this case, the internal pressure increased to 5.6 bar. The mixture was then cooled to 18° C. and the autoclave depressurized. 100 ml of water were added to the reaction mixture, which was stirred for a further 2 h, transferred to a separating funnel and the phases were separated. 163.7 g of an upper phase, 171.9 g of a middle phase and 47.6 g of a lower phase were obtained. The proportion of 2-fluoro-4-methyl-5-[(2,2,2-trifluoroethyl)sulfanyl]aniline in the upper phase was determined by quantitative HPLC (against external standard) as 15.6%. This corresponds to a yield of 71% starting from 5-amino-4-fluoro-2-methylbenzenethiol.

### Example 5: Synthesis of 2-fluoro-4-methyl-5-[(2,2,2-trifluoroethyl)sulfanyl]aniline in a solvent mixture of dimethyl sulfoxide and ethyl acetate (ratio 1:2)

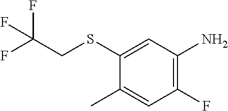

In a 500 mL autoclave composed of Hastelloy alloy were initially charged 23.58 g (150.0 mmol) of 5-amino-4-fluoro-2-methylbenzenethiol, 29.0 g (210 mmol) of potassium carbonate, 2.42 g (7.51 mmol) of tetra-n-butylammonium bromide, 150 ml of ethyl acetate and 73 mL of dimethyl sulfoxide. The autoclave was sealed, flushed several times with nitrogen, cooled to −10° C. and at this temperature 24.0 g (203 mmol) of 1,1,1-trifluoro-2-chloroethane were introduced. The autoclave was then heated to 60° C. and stirred at this temperature for 15 h (stirring speed: 300 rpm). In this case, the internal pressure increased to 0.7 bar. The mixture was then cooled to 18° C. and the autoclave depressurized. 100 ml of water were added to the reaction mixture and stirred for a further 30 min. The reaction mixture was discharged, the reactor washed twice with 50 mL of water and these wash solutions combined with the reaction mixture. The reaction mixture was transferred to a separating funnel and the phases were separated. 168.0 g of an upper phase and 316.9 g of a lower phase were obtained. The proportion of 2-fluoro-4-methyl-5-[(2,2,2-trifluoroethyl)sulfanyl]aniline in the upper phase was determined by quantitative HPLC (against external standard) as 14.4%. This corresponds to a yield of 68% starting from 5-amino-4-fluoro-2-methylbenzenethiol.

### Comparative example 1: Synthesis of 2-fluoro-4-methyl-5-[(2,2,2-trifluoroethyl)sulfanyl]aniline in pure dimethylformamide

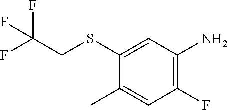

In a 300 mL autoclave composed of Hastelloy alloy were initially charged 8.12 g (90.4% purity, 46.7 mmol) of 5-amino-4-fluoro-2-methylbenzenethiol as a solution in 75 mL of dimethylformamide. 9.04 g (65.4 mmol) of potassium carbonate were added. The autoclave was then cooled with dry ice and 7.2 g (61 mmol) of 1,1,1-trifluoro-2-chloroethane were introduced. The autoclave was sealed and the internal pressure was increased to 10 bar by introducing nitrogen. The mixture was heated to 40° C. and stirred at this temperature for 16 h (stirring speed: 400 rpm). The autoclave was depressurized. The solvent was removed under reduced pressure and 50 mL of methyl tert-butyl ether and 100 mL of water were added to the residue. The phases were separated and the aqueous phase was extracted repeatedly with methyl tert-butyl ether. The combined organic phases were washed with water and subsequently dried with a drying agent (sodium sulfate or magnesium sulfate). The drying agent was removed by filtration and the solvent was removed under reduced pressure. 10.35 g of 2-fluoro-4-methyl-5-[(2,2,2-trifluoroethyl)sulfanyl]aniline were obtained as a brown oil at a purity by HPLC of 91.5 area percent and in a yield of 85%.

### Comparative example 2: Synthesis of 2-fluoro-4-methyl-5-[(2,2,2-trifluoroethyl)sulfanyl]aniline in pure ethyl acetate

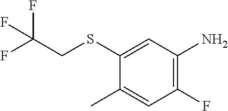

In a 300 mL autoclave composed of Hastelloy alloy were initially charged 8.70 g (90.4% purity, 50.0 mmol) of 5-amino-4-fluoro-2-methylbenzenethiol as a solution in 75 ml of ethyl acetate. 806 mg (2.50 mmol) of tetra-n-butylammonium bromide and 9.67 g (70.0 mmol) of potassium carbonate were added. The autoclave was then cooled with dry ice and 7.7 g (65 mmol) of 1,1,1-trifluoro-2-chloroethane were introduced. The autoclave was sealed and the internal pressure was increased to 10 bar by introducing argon. The mixture was heated to 60° C. and stirred at this temperature for 16 h (stirring speed: 600 rpm). The autoclave was depressurized and the contents poured into 200 mL of ice water with stirring. The mixture was stirred for 30 min and then the phases were separated. The aqueous phase was extracted three times in total with in each case 150 mL of methyl tert-butyl ether. The combined organic phases were washed twice with 30 mL of water each time and then with 30 ml of saturated sodium chloride solution. The organic phase was then dried with a drying agent (sodium sulfate or magnesium sulfate). The drying agent was removed by filtration and the solvent was removed under reduced pressure. 10.2 g of 2-fluoro-4-methyl-5-[(2,2,2-trifluoroethyl)sulfanyl]aniline were obtained as a brown oil at a purity by HPLC of 46.8 area percent and in a yield of 40%.

### Synthesis of 2-fluoro-4-methyl-5-[(2,2,2-trifluoroethyl)sulfanyl]aniline by alkylation with 2,2,2-trifluoroethyl methanesulfonate

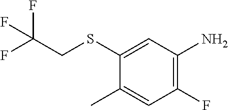

To 100 g of a 19% solution (121 mmol) of 5-amino-4-fluoro-2-methylbenzenethiol in ethyl acetate were added 1.95 g (6 mmol) of tetra-n-butylammonium bromide and 23.41 g (169 mmol) of potassium carbonate under a nitrogen atmosphere. Over a period of one hour, 22.6 g (127 mmol) of 2,2,2-trifluoroethyl methanesulfonate were then added dropwise. After completion of addition, the heating medium temperature was increased to 60° C. The mixture was stirred at 60° C. for 2 h and then 25 g of ethyl acetate were added. After a further 20 min, a further 20 g of ethyl acetate were added and the stirring speed was increased from 400 rpm to 700 rpm. After a total of 9 h 20 min stirring at 60° C., the reaction solution was cooled to room temperature. A further 50 g of ethyl acetate were then added and 100 g of water. The phases were separated and the organic phase was dried with sodium sulfate. The drying agent was removed by filtration and the solvent was then removed under reduced pressure. 29.3 g of 2-fluoro-4-methyl-5-[(2,2,2-trifluoroethyl)sulfanyl]aniline were obtained in a yield of 89%.

1. A method for preparing (2,2,2-trifluoroethyl)sulfanylaniline derivatives of formula (I): 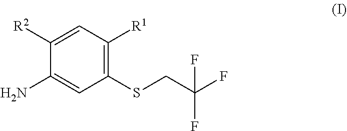
in which R¹ and R² are each independently (C₁-C₃) alkyl or halogen,
which is characterized in that a 3-aminobenzenethiol of formula (II): 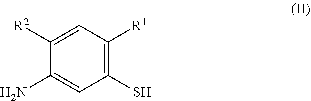
in which R¹ and R² have the definitions given above,
is reacted with 1,1,1-trifluoro-2-chloroethane in the presence of a base in a solvent mixture, wherein the solvent mixture comprises:
(i) a first solvent selected from N-methylpyrrolidone, N-ethylpyrrolidone, N-methylformamide, dimethylformamide, N,N-dimethylacetamide (DMAc), 1,3-dimethyl-2-imidazolidinone, tetramethylurea, sulfolane, dimethyl sulfoxide, acetonitrile, propionitrile, butyronitrile, polyethylene glycols, ethylene carbonate and propylene carbonate; and
(ii) a second solvent selected from tetrahydrofuran (THF), 1,2-dimethoxyethane (DME), 1,4-dioxane, diethyl ether, methyl tertbutyl ether (MTBE), tert-amyl methyl ether (TAME), 2-methyl-THF, cyclopentyl methyl ether, bis(2-methoxyethyl) ether, anisole, ethyl acetate, isopropyl acetate, butyl acetate, pentyl acetate, 3,3-dimethylbutanone, diethyl carbonate, dimethyl carbonate, toluene, xylenes and ethylbenzene.

2. The method according to claim 1, characterized in that the solvent mixture comprises:
(i) a first polar aprotic solvent selected from N-methylpyrrolidone, N-ethylpyrrolidone, N-methylformamide, dimethylformamide, N,N-dimethylacetamide (DMAc), 1,3-dimethyl-2-imidazolidinone, tetramethylurea, sulfolane, dimethyl sulfoxide, acetonitrile, propionitrile, butyronitrile, ethylene carbonate and propylene carbonate; and
(ii) a second less polar aprotic solvent selected from tetrahydrofuran (THF), 1,2-dimethoxyethane (DME), 1,4-dioxane, diethyl ether, methyl tertbutyl ether (MTBE), tert-amyl methyl ether (TAME), 2-methyl-THF, cyclopentyl methyl ether, bis(2-methoxyethyl) ether, anisole, ethyl acetate, isopropyl acetate, butyl acetate, pentyl acetate, 3,3-dimethylbutanone, toluene, xylenes and ethylbenzene.

3. The method according to claim 1, characterized in that R¹ and R² are each independently fluorine, chlorine or methyl.

4. The method according to claim 3, characterized in that R¹ and R² are each independently fluorine or methyl.

5. The method according to claim 4, characterized in that R¹ is methyl and R² is fluorine.

6. The method according to claim 1, characterized in that the first solvent is selected from N-methylpyrrolidone, N-methylformamide, dimethylformamide, N,N-dimethylacetamide (DMAc), sulfolane, dimethyl sulfoxide and polyethylene glycol of molar masses of 200-800 g/mol (polyethylene glycol 200-800).

7. The method according to claim 1, characterized in that the second solvent is selected from tetrahydrofuran (THF), dimethyl ether (DME), 1,4-dioxane, 2-methyl-THF, ethyl acetate, isopropyl acetate, butyl acetate and pentyl acetate.

8. The method according to claim 6, characterized in that the first solvent is selected from N-methylpyrrolidone, dimethylformamide, N,N-dimethylacetamide (DMAc), dimethyl sulfoxide and polyethylene glycol 400.

9. The method according to claim 7, characterized in that the second solvent is selected from tetrahydrofuran (THF), ethyl acetate and isopropyl acetate.

10. The method according to claim 1, characterized in that a ratio of the first solvent to the second solvent is in a range from 20:1 to 1:20.

11. The method according to claim 1, characterized in that the base is selected from alkali metal hydroxide, alkaline earth metal hydroxide, ammonium or alkylammonium hydroxide, sodium hydride, calcium hydride, alkali metal or alkaline earth metal alkoxide, alkali metal or alkaline earth metal carbonate, ammonia, primary, secondary or tertiary alkyl, aryl or aralkylamine, amidine and pyridine.

12. The method according to claim 11, characterized in that the base is selected from sodium hydroxide, potassium hydroxide, sodium carbonate and potassium carbonate.

13. The method according to claim 12, characterized in that the base is selected from sodium carbonate and potassium carbonate.

14. The method according to claim 1, characterized in that the base is used anhydrous or as an aqueous solution.

15. The method according to claim 1, characterized in that a molar ratio of base to thiol of the formula (II) is in a range from 0.9:1 to 5:1.

16. The method according to claim 1, characterized in that said method is carried out at a temperature between 0° C. and 100° C.

17. The method according to claim 1, characterized in that said method is carried out at a pressure between 0 bar and 20 bar above atmospheric pressure.

18. The method according to claim 1, characterized in that said method is carried out in the presence of a phase transfer catalyst.

19. The method according to claim 18, wherein the phase transfer catalyst is tetra-n-butylammonium bromide.
